(19) United States
(12) Patent Application Publication
(10) Pub. No.: US 2022/0370444 A1
(43) Pub. Date: Nov. 24, 2022

(54) Compositions of Clofazimine, Combinations Comprising Them, Processes for Their Preparation, Uses and Methods of Treatment Comprising Them

(71) Applicant: MannKind Corporation, Westlake Village, CA (US)

(72) Inventors: Thomas Hofmann, Doylestown, PA (US); Stefan Ufer, Raleigh, NC (US); Kevin Stapleton, Laguna Beach, CA (US)

(73) Assignee: MannKind Corporation, Danbury, CT (US)

(21) Appl. No.: 17/755,425

(22) PCT Filed: Nov. 1, 2020
(86) PCT No.: PCT/US2020/058447

§ 371 Date: Apr. 28, 2022

Related U.S. Application Data
(60) Provisional application No. 62/931,437, filed on Nov. 6, 2019.

(51) Int. Cl. A61K 31/498; A61K 9/00; A61K 9/16; A61K 31/47; A61K 31/606; A61K 31/546; A61K 31/7036; A61K 31/7048; A61K 31/4965; A61K 31/496; A61K 31/4709; A61K 31/5383

(52) U.S. Cl. CPC A61K 31/498; A61K 9/0075; A61K 9/1617; A61K 9/1623; A61K 31/47; A61K 31/606; A61K 31/546; A61K 31/7036; A61K 31/7048; A61K 31/4965; A61K 31/496; A61K 31/4709; A61K 31/5383

(57) ABSTRACT

The present invention relates to pharmaceutical compositions for inhalation comprising a therapeutically effective dose of clofazimine wherein the clofazimine is provided in the form of dry powder, and processes for their preparation. Furthermore, the present invention provides pharmaceutical combinations comprising clofazimine in the form of an aerosol for pulmonary inhalation. The combinations and compositions provided by the present invention may be used in the treatment and/or prophylaxis of pulmonary infections caused by mycobacteria and other gram-positive bacteria, and of pulmonary fungal infections.

This application is a national stage application of PCT/US2020/058447, filed Nov. 1, 2020, which claims the benefit of U.S. Provisional Application No. 62/931,437, filed Nov. 6, 2019, the entirety of their contents of which are incorporated herein by reference.

### FIELD OF THE INVENTION

The present invention relates to pharmaceutical compositions for inhalation comprising a therapeutically effective dose of clofazimine, wherein the clofazimine is provided in the form of a dry powder; processes for their preparation; and uses and methods of treatment comprising them. Furthermore, the present invention provides pharmaceutical combinations comprising clofazimine in the form of an aerosol for pulmonary inhalation.

The combinations and compositions provided by the present invention may be used in the treatment and/or prophylaxis of pulmonary infections caused by mycobacteria and other gram-positive bacteria, and of pulmonary fungal infections.

### BACKGROUND OF THE INVENTION

Clofazimine is an extremely hydrophobic riminophenazine antibiotic (Log P=7.66) with anti-mycobacterial and anti-inflammatory activities and was originally described in 1957. Its structural formula is as follows:

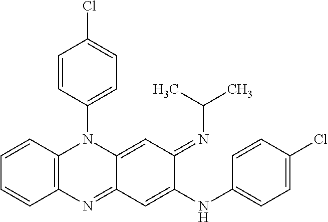 [SMILES: CC(C)/N=c1\cc2n(-c3ccc(Cl)cc3)c3ccccc3nc-2cc1Nc1ccc(Cl)cc1]

The exact mechanism through which clofazimine exerts its antimicrobial effect is unknown. However, it is known to bind preferentially to mycobacterial DNA, thereby inhibiting DNA replication and cell growth. Other suggested mechanisms of action include membrane damage/destabilization, generation of membrane-destabilizing lysophospholipids, interference of potassium transport, and/or intracellular redox cycling. While impressively active against Mycobacterium tuberculosis (MTB) in vitro, including multidrug-resistant strains, clofazimine, until recently, was generally considered to be ineffective in the treatment of pulmonary tuberculosis (see, for example, Cholo M et al., J Antimicrob Chemother, 2012 February 67(2):290-8).

Clofazimine is one of the three principal drugs recommended by the World Health Organization for the treatment of leprosy which is caused by Mycobacterium leprae and has been increasingly used for the treatment of other mycobacterial infections such as drug resistant tuberculosis and infections caused by nontuberculous mycobacteria (NTM) in recent years.

Clofazimine has been classified as a Biopharmaceutics Classification System (BCS) class II drug as it is practically insoluble in water and shows high membrane permeability.

To overcome the problems associated with poor oral absorption and poor bioavailability of drugs, various strategies have been applied such as micronization, nanonization, supercritical fluid re-crystallization, spray freeze drying into liquid, solid dispersions and solutions in optimizing oral dosage forms.

Being classified as a BCS class II drug, clofazimine is generally considered an ideal candidate for the formulation into solid dispersions for improvement of oral bioavailability (see, for example, Bhusnure et al. IJRPC 2014, 4(4), 906-918).

In line with this, because of its lipophilicity, clofazimine is generally administered as a microcrystalline suspension in an oil-wax base to improve oral absorption. The absorption in humans after oral administration varies considerably (45-62%). Adverse effects of clofazimine are dose related and primarily affect the skin, eyes, gastrointestinal tract, and QT elongation Side effects include the development of reddish-brown discoloration of the skin and conjunctiva and are gradually reversible on cessation. They are the result of chronic systemic accumulation.

Mycobacterium is a genus Actinobacteria, with its own genus, Mycobacteriaceae. Mycobacteria have characteristic rod-like shapes and waxy outer coats.

As such, Mycobacteria can be divided into three groups:
Mycobacterium tuberculosis complex—causative pathogen of tuberculosis
Mycobacterium leprae—causative pathogen of leprosy
Nontuberculous mycobacteria (NTM) which encompass all other mycobacteria that are not M. tuberculosis or M. leprae, including Mycobacterium abscessus complex (MABSC), Mycobacterium avium complex (MAC).

Tuberculosis (TB) is an infectious disease caused by Mycobacterium tuberculosis complex bacteria. As one of the oldest documented infectious agents in humans, TB remains a significant cause of mortality and morbidity worldwide, with an estimated 10.4 million new cases of TB infection, and 1.4 million people killed by active TB disease in 2015 (see, for example, World Health Organization (WHO) Global Tuberculosis Report 2016). In addition to the high prevalence and mortality rates, the incidence of multi-drug resistant tuberculosis (MOR-TB) is a growing concern, with 580,000 patients presenting with a drug-resistant TB infection in 2015. Co-morbidities, such as human immunodeficiency virus (HIV), complicate treatment, and were responsible for 1.2 million cases of TB in 2015.

To treat multi-drug resistant (MOR) infections, the WHO has recommended implementing a 9 to 12-month treatment regimen of second-line anti-TB drugs. These regimens, such as the 9 to 12 month Bangladesh regimen, treat MOR-TB with a combination of gatifloxacin, ethambutol, pyrazinamide, and clofazimine, which led to a relapse-free cure in 87.9% of patients (see, for example, Sotgiu, G, et al., “Applicability of the shorter ‘Bangladesh regimen’ in high multidrug-resistant tuberculosis settings”, International Journal of Infectious Diseases (2017) 56 190-193).

Other studies investigating shortened TB treatments demonstrated that clofazimine had no clinical benefit after two weeks of oral administration (see, for example, Oiacon, A. H., et al., “Bactericidal Activity of Pyrazinamide and Clofazimine Alone and in Combinations with Pretomanid and Bedaquiline”, American Journal of Respiratory and Critical Care Medicine (2015), 191 (8), 943-953). The lack of activity was attributed to low bioavailability of the drug, as it was theorized to bind to circulating serum proteins with a high affinity. There, despite the fact that clofazimine has been empirically demonstrated to be effective for the treatment of MOR-TB, and extensively-drug resistant TB (XOR-TB), its poor bioavailability after systemic administration appears to limit its biological activity over short duration therapies (see, for example, Swanson, R.V., et al., “Pharmacokinetics and Pharmacodynamics of Clofazimine in a Mouse Model of Tuberculosis”, Antimicrobial Agents and Chemotherapy (2015), 59 (6), 3042-3051).

It is known that treatment of lung infections with inhaled antibiotics results in higher drug concentrations in the lungs and reduced adverse effects compared to systemic delivery (see, for example, Touw, O. J., et al., “Inhalation of antibiotics in cystic fibrosis”, European Respiratory Journal (1995), 8, 1594-1604), which result in increased biological activity and efficacy (see, for example, Hickey, A. J., “Inhaled drug treatment for tuberculosis: Past progress and future prospects”, Journal of Controlled Release, (2016), 240, 127-134). In vivo mouse models have demonstrated that aerosolized administration of clofazimine shows significant improvement in bacilli clearance in TB-infection models compared to oral administration of clofazimine only 28 days after treatment initiation (see, for example, Verma, R. K., et al., “Inhaled microparticles containing clofazimine are efficacious in treatment of experimental Tuberculosis in Mice”, Antimicrobial Agents and Chemotherapy (2013), 57 (2), 1050-1052). This improved efficacy over a short duration is likely due to the direct delivery of clofazimine to the site of infection in the lungs resulting in higher clofazimine concentration in the pulmonary macrophages within the tuberculosis granulomas.

Accordingly, the use of an aerosolized administration of clofazimine in patients with MOR TB, or XOR-TB infections should further improve patient treatment outcomes, and may shorten the duration of current treatment regimens.

The group of nontuberculous mycobacteria (NTM), formerly called atypical or ubiquitous mycobacteria, contains over 150 species. NTM can be found ubiquitously in nature and show a broad diversity. They can be detected in soil, ground and drinking water as well as in food like pasteurized milk or cheese. In general, NTM are considered to be less pathogenic. Nevertheless, they can cause severe illness in humans, especially in immune compromised persons or those who suffer from previous pulmonary diseases. Currently NTM are classified according to their growth rate and are divided into slow-growing (SGM) and rapid-growing (RGM) mycobacteria.

The slow growing Mycobacterium avium complex (MAC) comprises the species Mycobacterium avium, Mycobacterium chimaera and Mycobacterium intracellulare that are among the most important and most frequent pathogenic NTM. Just like Mycobacterium kansasii, Mycobaceterium malmoense, Mycobacterium xenopi,

Mycobacterium. simiae, Mycobacterium abscessus, Mycobacterium gordonae, Mycobacterium fortuitum, and Mycobacterium chelonae, they mostly cause pulmonary infections. Mycobacterium marinum is responsible for skin and soft tissue infections like aquarium granuloma.

In particular, RGM cause serious, life-threatening chronic lung diseases and are responsible for disseminated and often fatal infections. Infections are typically caused by contaminated materials and invasive procedures involving catheters, non-sterile surgical procedures or injections and implantations of foreign bodies. Exposure to shower heads and jacuzzis has also been reported as risks for infections. NTM typically cause opportunistic infections in patients with chronic pulmonary diseases such as chronic obstructive pulmonary disease (COPD), cystic fibrosis (CF), and other immune compromised patients.

In recent years, the rapidly growing (RGM) Mycobacterium abscessus group strains (Mycobacterium abscessus complex, MABSC) comprising the subspecies Mycobacterium abscessus subsp. abscessus (M. a. abscessus), Mycobacterium abcessus bolletii. and Mycobacterium abscessus massiliense have emerged as important human pathogens and are associated with significantly higher fatality rates than any other RGM.

Mycobacterium abscessus infection in CF patients are particularly problematic, as it results in enhanced pulmonary destruction and is often impossible to treat with failure rates as high as 60-66%. (see, for example, Obregon-Henao A et al, Antimicrobial Agents and Chemotherapy, November 2015, Vol 59, No 11, p. 6904-6912; Qvist,T., Pressler,T., Hoiby,N. and Katzenstein,T L., “Shifting paradigms of nontuberculous mycobacteria in cystic fibrosis”, Respiratory Research (2014), 15(1):pp.41-47).

Human infection with NTM became of greater relevance with the emergence of the human acquired immune deficiency syndrome pandemic. Mycobacteria from Mycobacterium avium complex (MAC) were identified as the major cause of opportunistic infections in patients infected with the human immunodeficiency virus (HIV).

Several species of NTM are known to form biofilms. Biofilms are microcolonies of bacteria embedded in the extracellular matrix that provide stability and resistance to human immune mechanisms. In recent years, some species of NTM have been shown to form biofilms that enhance resistance to disinfectants and antimicrobial agents. Biofilm assembly proceeds through several phases, including reversible attachment, irreversible attachment, biofilm formation via bacterial aggregation, organization, and signaling, and finally dispersion. During this process, bacteria develop a matrix containing extracellular polymeric substances (EPS), such as polysaccharides, lipids and nucleic acids, to form a complex three-dimensional structure (see, for example, Sousa S. et al., International Journal of Mycobacteriology 4 (2015), 36-43). Specifically, mycobacterial EPS differ in nature from other biofilms, as mycobacteria do not produce exopolysaccharides (see, for example, Zambrano MM, Kolter R. Mycobacterial biofilms: a greasy way to hold it together. Cell. 2005}. Mycobacterial biofilms vary between species, but can contain mycolic acids, glycopeptidolipids, mycolyl-diacylglycerols, lipooligosaccharides, lipopeptides, and extracellular DNA (Overview and original research from: Rose S J, Babrak L M, Bermudez L E (2015) Mycobacterium avium Possesses Extracellular DNA that Contributes to Biofilm Formation, Structural Integrity, and Tolerance to Antibiotics . . . PLoS ONE). The assembly in biofilms is known to enhance resistance to antimicrobial agents (see, for example, Faria S. et al., Journal of Pathogens, Vol 2015, Article ID 809014).

Delivery of aerosolized liposomal amikacin/inhaled amikacin solution nebulized by a jet nebulizer as a novel approach for treatment of NTM pulmonary infection has been suggested (Rose S. et al, 2014, PLoS ONE, Volume 9, Issue 9, e108703, and Olivier K. et al, Ann Am Thorac Soc Vol 11, No 1, pp. 30-35) as well as inhalation of anti-TB drugs dry powder microparticles for pulmonary delivery (Cholo Met al., J Antimicrob Chemother. 2012 February; 67(2):290-8 and Fourie B. and Nettey O., 2015 Inhalation Magazine, Verma 2013 Antimicrob Agents Chemother}.

Multiple combination regimens with inhaled amikacin following initial treatment with parenteral aminoglycosides, tigecycline and other promising oral antibiotics such as linezolid, delamanid, and bedaquiline, and surgical intervention in selected cases have shown promising results in the treatment of NTM lung disease (Lu Ryu et al., Tuberc Respir Dis 2016;79:74-84). However, the increasing incidence and prevalence of NTM infections, in particular NTM lung disease and the limited treatment options necessitate the development of novel dosage forms/pharmaceutical formulation enhancing the bioavailability of the currently used antibiotics such as clofazimine. Inhalation may enhance efficacy and reduce adverse effects compared to oral and parenteral therapies.

Combinations of clofazimine and amikacin have been shown to act synergistically in vitro against both Mycobacterium abscessus and Mycobacterium avium (see, for example, van Ingen, J., et al., “In Vitro Synergy between Clofazimine and Amikacin in Treatment of Nontuberculous Mycobacterial Disease”, Antimicrobial Agents and Chemotherapy 56 (12), 6324-6327 (2012)). Further, synergy has been shown with combinations of clofazimine and bedaquiline used against Mycobacterium tuberculosis (see, for example, Cokol, M. et al., “Efficient Measurement and factorization of high-order drug interactions in Mycobacterium tuberculosis”, Sciences Advances 2017:3:e170881, 11 Oct. 2017).

Synergy has also been shown for a clofazimine/bedaquiline combination against the nontuberculous bacterium Myocbacterium abscessus (Ruth, M. M. et al., “A Bedaquiline/Clofazimine Combination Regimen Might Add Activity to the Treatment of Clinically Relevant Non-Tuberculous Mycobacteria”, Journal of Antimicrobial Chemotherapy (2019), doi.org/10.1093/jac/dky526).

Fungal pathogens have emerged as a leading cause of human mortality. Current estimates suggest death due to invasive fungal infections is on par with more well-known infectious diseases such as tuberculosis. Candida albicans, Cryptococus neoformans, and Aspergillis fumigatus represent the most prevalent fungal pathogens of humans. Each of these species is responsible for hundreds of thousands of infections annually with

unacceptably high mortality rates due to poor diagnostics and limited treatment options. Clofazimine has been shown to exhibit efficacy as a combination agent against multiple fungi. (see, for example, Robbins, N., et al., “An Antifungal Combination Matrix Identifies a Rich Pool of Adjuvant Molecules that Enhance Drug Activity against Diverse Fungal Pathogens”, Cell Reports 13, 1481-1492, Nov. 17, 2015). Fungi also play a role as commensals, colonizers and/or pathogens in cystic fibrosis (see, for example, Chotirmall, S. H. and McElvaney, N. G., “Fungi in the cystic fibrosis lung: Bystanders or pathogens?”, The International Journal of Biochemistry & Cell Biology 52 (2014), 161-173.

### SUMMARY OF THE INVENTION

According to one aspect of the invention, a pulmonary mycobacterial infection is treated with clofazimine delivered directly to the lungs via oral inhalation. The dose delivered to the patient is lower than the corresponding oral dose.

One aspect of this invention is the delivery of between 10 and 20 mg of clofazimine to the patient's lungs. The clofazimine can be in the form of a neat drug, or a pharmaceutically acceptable derivative or salt.

There are a number of embodiments that can be used to deliver said amount of drug to patients via aerosol. One embodiment is a dry powder inhaler.

One skilled in the art can envision many embodiments that vary slightly in description, but still have the same therapeutic effect of delivering between 10 mg and 20 mg of clofazimine to the lungs.
1. Alternate forms of clofazimine: the powder could be manufactured with a pharmaceutically acceptable derivative, polymorph, or salt of clofazimine
2. Use of alternative inhalers: the formulation could be adapted for use with any dry
powder inhaler, including other capsule based devices, blister strip inhalers, reservoir inhalers, disposable inhalers, and re-usable inhalers.
3. Alternative particle sizes: Each inhaler has a different resistance to air flow, with higher resistance inhalers resulting in lower inhalation flow rates. Choice of an inhaler with a higher resistance (lower inhalation flow rate) enables the use of larger particle sizes (up to 10 μm) for effective lung delivery
4. Alternative formulation components: Numerous grades of lactose are available for use in inhalation formulations that vary in size and geometry. Small lactose particles can also be pre-blended to assist in dispersion. Lactose may be replaced with a physiologically acceptable pharmacologically inert solid carrier.
Additional excipients such as phospholipids, salts, surfactants or polymers may be added to assist in aerosol dispersion.
5. Alternative formulation forms: Alternatively, the clofazimine and excipients could be dissolved in a solvent or solvents and spray dried.

The aerosolization of the compositions of the invention by an appropriate inhaler provides significantly increased delivery of the aerosolized clofazimine into the lower lung (i.e. to the bronchi, bronchioli, and alveoli of the central and lower peripheral lungs), thereby substantially enhancing the therapeutic efficacy.

The inhalation device should, moreover, preferably be further adapted for localized pulmonary delivery of an aerosol having an optimal particle size distribution for homogenous deposition in the lower lung.

The invention therefore provides for an aerosol having aerosol particles of sizes that facilitate delivery to the alveoli and bronchiole. A suitable aerodynamic particle size for targeting the alveoli and bronchiole is between 1 and 5 μm. Particles larger than that are selectively deposited in the upper lungs, namely bronchi and trachea and in the mouth and throat, i.e. oropharyngeal area. Accordingly, the inhalation device is adapted to produce an aerosol having a mass median aerodynamic diameter (MMAD) in the range from about 1 to about 5 μm, and preferably in the range from about 1 to about 3 μm. In a further embodiment, the particle size distribution is narrow and has a geometric standard deviation (GSD) of less than about 2.5.

### DETAILED DESCRIPTION OF THE INVENTION

The present invention is based on the unexpected discovery that by pulmonary aerosol administration of clofazimine, lower (i.e. deeper) lung deposition of the active agent can be achieved, thereby significantly increasing the bioavailability of the extremely hydrophobic BCS class II agent, which results in significantly increased therapeutic efficacy coupled with reduced systemic side effects.

In another aspect, this finding leads to the provision of an improved antibiotic therapy for infections caused by mycobacteria and gram-positive bacteria, in particular of pulmonary infections with NTM, such as opportunistic infections in cystic fibrosis, chronic obstructive pulmonary disease and immune compromised patients such as HIV patients.

The present invention, moreover, aims at overcoming systemic side effects of established oral treatment regimens for pulmonary infections with gram-positive bacteria, in particular TB and NTM infections of the lungs as well as at the reduction of dose and of duration of treatment with clofazimine.

It is understood by the person of skill in the art that the present application also discloses each and any combination of the individual features disclosed herein.

### Definitions

The term “pharmaceutically acceptable salt” refers to salts that retain the biological effectiveness and properties of the compounds of this invention and, which are not biologically or otherwise undesirable. In many cases, the compounds of this invention are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, naphtoic acid, oleic acid, palmitic acid, pamoic (emboic) acid, stearic acid, glycolic acid, pyruvic acid, oxalic acid, maleic acid, malonic acid, succinic acid, fumaric acid, tartaric acid, citric acid, ascorbic acid, glucoheptonic acid, glucuronic acid, lactic acid, lactobionic acid, tartaric acid, benzoic acid, cinnamic acid, mandelic acid, methanesulfonic acid, ethanesulfonic acid, p-toluenesulfonic acid, salicylic acid, and the like.

Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, histidine, arginine, lysine, benethamine, N-methyl-glucamine, and ethanolamine. Other acids include dodecylsufuric acid, naphthalene-1,5-disulfonic acid, naphthalene-2-sulfonic acid, and saccharin.

In accordance with the present invention, apart from the free base, the use of the methanesulfonic acid, maleic acid, isonicotinic acid, nicotinic acid, malonic acid, and salicylic acid salts, and in particular of clofazimine mesylate is preferred.

By the term “pharmaceutically acceptable derivative” as used herein, for example, compounds disclosed in US 9,540,336 are meant, the disclosure of US 9,540,336 is incorporated herein in its entirety. In addition, derivatives are meant as described in Lu, Y., Zhen, M., Wang, B., Fu, L., Zhao, W., Li, P., Xu, J., Zhu, H., Jin, H., Yin, D., Huang, H., Upton, A M. and Ma, Z., “Clofazimine Analogs with Efficacy against experimental Tuberculosis and reduced Potential for Accumulation” Antimicrobial Agents and Chemotherapy (2011), 55(11): pp.5185-5193. Additionally, the term, “pharmaceutically acceptable derivative” of a compound is, for example, a prodrug of said compound. In general, a prodrug is a derivative of a compound which, upon administration, is capable of providing the active form of the compound. Such derivatives, for example, may be an ester or amide of a carboxyl group, a carboxyl ester of a hydroxyl group, or a phosphate ester of a hydroxyl group.

By “patient” is meant a mammal, preferably a human, in need of the prophylaxis and or the treatments as described herein.

By “therapeutically effective amount”, “therapeutically effective dose”, or “pharmaceutically effective amount” is meant an amount of clofazimine, or a pharmaceutically acceptable salt or derivative thereof, as disclosed for this invention, which has a therapeutic effect. The doses of clofazimine which are useful in treatment are therapeutically effective amounts. Thus, as used herein, a therapeutically effective amount means those amounts of clofazimine which produce the desired therapeutic effect as judged by clinical trial results and/or model animal infection studies.

The amount of the clofazimine and daily dose can be routinely determined by one of skill in the art, and will vary, depending on several factors, such as the particular microbial strain involved. This amount can further depend upon the patient's height, weight, sex, age and medical history. For prophylactic treatments, a therapeutically effective amount is that amount which would be effective to prevent a microbial infection.

A “therapeutic effect” relieves, to some extent, one or more of the symptoms of the infection, and includes curing an infection. “Curing” means that the symptoms of active infection are eliminated, including the total or substantial elimination of excessive members of viable microbe of those involved in the infection to a point at or below the threshold of detection by traditional measurements. However, certain long-term or permanent effects of the infection may exist even after a cure is obtained (such as extensive tissue damage). As used herein, a “therapeutic effect” is defined as a statistically significant reduction in bacterial load in a host, emergence of resistance, or improvement in infection symptoms as measured by human clinical results or animal studies.

“Treat”, “treatment”, or “treating” as used herein refers to administering a pharmaceutical composition/combination for prophylactic and/or therapeutic purposes.

The term “prophylactic treatment” or “prophylaxis” refers to treating a patient who is not yet infected, but who is susceptible to, or otherwise at risk of, a particular infection. The term “therapeutic treatment” refers to administering treatment to a patient already suffering from an infection. Thus, in preferred embodiments, treating is the administration to a mammal (either for therapeutic or prophylactic purposes) of therapeutically effective amounts of clofazimine.

Unless stated otherwise herein, the term “inhalation” is meant to refer to pulmonary inhalation.

Unless stated otherwise herein, the term “infection” as used herein is meant to refer to pulmonary infections.

Unless otherwise stated, the term “substantially” when used to refer to the purity of a compound, indicates a purity of compound of 95% or greater purity.

Unless otherwise stated, the term “appropriate particle size” refers to a particle size of clofazimine in a composition, or a composition that provides the desired therapeutic effect when administered to a patient.

Unless otherwise stated, the term “appropriate concentration” refers to a concentration of a component in a composition or combination which provides a pharmaceutically acceptable composition or combination.

### Pharmaceutical Compositions and Combinations

Clofazimine has been shown to exist in at least four polymorphic forms (see, for example, Bannigan, et al., “Investigation into the Solid and Solution Properties of Known and Novel Polymorphs of the Antimicrobial Molecule Clofazimine”, Cryst. Growth Des. 2016, 16 (12), pp. 7240-7250). Clofazimine can exist in a triclinic form FI, a monoclinic form FII, and an orthorhombic form FIII. A further form FIV has also been seen only at high temperatures.

Accordingly, in a further embodiment of the invention a pharmaceutical composition is provided comprising: (a) a therapeutically effective dose of clofazimine; wherein the clofazimine is provided in the form of particles in a dry powder, and wherein the particles of clofazimine have a median size of less than 5 μm and a D90 of less than 6 μm, preferably a median size of less than 2 μm and a D90 of less than 3 μm, and wherein the clofazimine is provided in a polymorphic form or forms selected from triclinic form FI, monoclinic form FII and orthorhombic form FIII and mixtures of such forms. In a preferred embodiment, the clofazimine is provided substantially in orthorhombic form FIII.

In another embodiment, a pharmaceutical composition according to any one of the composition embodiments described herein is provided which is for use in combination with an agent for dispersing and/or destruction of biofilm, with mucolytic and/or mucoactive agents, and/or agents that reduce biofilm formation selected from metaperiodate, sodium dodecyl sulfate, sodium bicarbonate, tromethamine, silver nano particles, bismuth thiols, ethylene diamine tetraacetic acid, gentamicin loaded phosphatidylcholine-decorated gold nanoparticles, chelators, cis-2-decenoic acid, D-amino acids, D-enantiomeric peptides, gallium mesoporphyrin IX, gallium protoporphyrin IX, curcumin, patulin, penicillic acid, baicalein, naringenin, ursolic acid, asiatic acid, corosolic acid, fatty acids, host defense peptides, and antimicrobial peptides. In another embodiment, the composition for the use is administered before, simultaneously, or subsequently to the administration of an agent selected from bedaquiline or a pharmaceutically acceptable salt or derivative thereof, cefoxitine, amikacin, clarithromycin, pyrazinamide, rifampin, moxifloxacin, levofloxacin, and para-amino salicylate, and mixtures thereof.

In another embodiment, a pharmaceutical combination according to any of the combination embodiments described herein is provided which is for use in combination with an agent for dispersing and/or destruction of biofilm, with mucolytic and/or mucoactive agents, and/or agents that reduce biofilm formation selected from metaperiodate, sodium dodecyl sulfate, sodium bicarbonate, tromethamine, silver nano particles, bismuth thiols, ethylene diamine tetraacetic acid, gentamicin loaded phosphatidylcholine-decorated gold nanoparticles, chelators, cis-2-decenoic acid, D-amino acids, D-enantiomeric peptides, gallium mesoporphyrin IX, gallium protoporphyrin IX, curcumin, patulin, penicillic acid, baicalein, naringenin, ursolic acid, asiatic acid, corosolic acid, fatty acids, host defense peptides, and antimicrobial peptides. In another embodiment, the combination for the use is used to administer a composition of the present invention before, simultaneously, or subsequently to the administration of an agent selected from bedaquiline or a pharmaceutically acceptable salt or derivative thereof, cefoxitine, amikacin, clarithromycin, pyrazinamide, rifampin, moxifloxacin, levofloxacin, and para-amino salicylate, and mixtures thereof. In another embodiment, the composition is administered before, simultaneously or subsequently to the administration of an agent selected from bedaquiline or a pharmaceutically acceptable salt or derivative thereof, and amikacin, and mixtures thereof. In a further embodiment, the composition is administered before, simultaneously or subsequently to the administration of bedaquiline or a pharmaceutically acceptable salt or derivative thereof.

In another embodiment, a pharmaceutical composition according to any one of the composition embodiments as described herein is provided for use in the treatment and/or prophylaxis of a pulmonary infection caused by mycobacteria or other gram-positive bacteria. In a further embodiment, the infection is caused by a species of the genus mycobacterium selected from nontuberculous mycobacteria and Mycobacterium tuberculosis complex, and a combination thereof. In a further embodiment, the nontuberculous mycobacteria are selected from Mycobacterium avium, Mycobacterium intracellulare, Mycobacterium abscessus, and Mycobacterium leprae, and a combination thereof. In another embodiment, the infection is an opportunistic infection, selected from MAC pulmonary disease and nontuberculous infection, in a patient with cystic fibrosis, chronic obstructive pulmonary or acquired immune deficiency syndrome. In another embodiment, the infection is an opportunistic nontuberculous mycobacteria infection in patients with cystic fibrosis. In another embodiment, the composition for the use is administered before, simultaneously, or subsequently to the administration of an agent selected from bedaquiline or a pharmaceutically acceptable salt or derivative thereof, cefoxitine, amikacin, clarithromycin, pyrazinamide, rifampin, moxifloxacin, levofloxacin, and para-amino salicylate, and mixtures thereof. In another embodiment, the composition is administered before, simultaneously or subsequently to the administration of an agent selected from bedaquiline or a pharmaceutically acceptable salt or derivative thereof, and amikacin, and mixtures thereof. In a further embodiment, the composition is administered before, simultaneously or subsequently to the administration of bedaquiline or a pharmaceutically acceptable salt or derivative thereof.

In another embodiment a pharmaceutical combination according to any of the combination embodiments as described herein is provided for use in the treatment and/or prophylaxis of a pulmonary infection caused by mycobacteria or other gram-positive bacteria. In a further embodiment, the infection is caused by a species of the genus mycobacterium selected from nontuberculous mycobacteria and Mycobacterium tuberculosis complex, and a combination thereof. In a further embodiment, the nontuberculous mycobacteria are selected from Mycobacterium avium, Mycobacterium intracellulare, Mycobacterium abscessus, and Mycobacterium leprae, and a combination thereof. In another embodiment, the infection is an opportunistic infection, selected from MAC pulmonary disease and nontuberculous infection, in a patient with cystic fibrosis, chronic obstructive pulmonary or acquired immune deficiency syndrome. In another embodiment, the infection is an opportunistic nontuberculous mycobacteria infection in patients with cystic fibrosis. In another embodiment, the combination for the use is used to administer a composition of the present invention before, simultaneously, or subsequently to the administration of an agent selected from bedaquiline or a pharmaceutically acceptable salt or derivative thereof, cefoxitine, amikacin, clarithromycin, pyrazinamide, rifampin, moxifloxacin, levofloxacin, and para-amino salicylate, and mixtures thereof. In another embodiment, the combination for the use is used to administer a composition of the present invention before, simultaneously or subsequently to the administration of an agent selected from bedaquiline or a pharmaceutically acceptable salt or derivative thereof, and amikacin, and mixtures thereof. In another embodiment, the combination for the use is used to administer a composition of the present invention before, simultaneously or subsequently to the administration of bedaquiline or a pharmaceutically acceptable salt or derivative thereof.

In another embodiment, a system for use in providing antibiotic activity when treating or providing prophylaxis against a pulmonary infection caused by mycobacteria or other gram-positive bacteria is provided wherein the system comprises: 1) an aerosolized pharmaceutical combination comprising: (a) a therapeutically effective dose of clofazimine; and 2) a dry powder inhaler, wherein the clofazimine is present in the form of a dry powder, and wherein the aerosol particles produced by the system have a mass median aerodynamic diameter of 1 to 5 μm.

In a further embodiment, a pharmaceutical composition according to any one of composition embodiments described herein is provided, for use in the treatment and/or prophylaxis of pulmonary fungal infections or clostridium difficile, or a combination thereof.

In another embodiment, a pharmaceutical composition according to any one of composition embodiments described herein is provided, for use in the treatment and/or prophylaxis of pulmonary fungal infections. In a further embodiment, the pulmonary fungal infection is candida albicans or aspergilus fumigatus, or a combination thereof.

In a further embodiment, a pharmaceutical combination according to any one of the combination embodiments described herein is provided, for use in the treatment and/or prophylaxis of pulmonary fungal infections or clostridium difficile, or a combination thereof. A pharmaceutical combination according to any one of combinations embodiments described herein is provided, for use in the treatment and/or prophylaxis of pulmonary fungal infections. In a further embodiment, the pulmonary fungal infection is candida albicans or aspergilus fumigatus, or a combination thereof.

In another embodiment, a method of treatment or prophylaxis of a pulmonary infection is provided, in a patient in need thereof, comprising administering by inhalation a composition according to any one the composition embodiments described herein. In another embodiment, the infection is caused by a species of the genus mycobacterium selected from nontuberculous mycobacteria and Mycobacterium tuberculosis complex, and a combination thereof. In a further embodiment, the nontuberculous mycobacterium is selected from Mycobacterium avium, Mycobacterium intracellulare, Mycobacterium abscessus, and Mycobacterium leprae, and a combination thereof. In a further embodiment, the infection is an opportunistic infection, selected from MAC pulmonary disease and nontuberculous infection, in a patient with cystic fibrosis, chronic obstructive pulmonary disease or acquired immune deficiency syndrome. In another embodiment, the infection is an opportunistic nontuberculous mycobacteria infection in a patient with cystic fibrosis.

In a further embodiment, a method of treatment or prophylaxis of a pulmonary infection is provided caused by mycobacteria or other gram-positive bacteria, in a patient in need thereof, comprising administering by inhalation a composition according to any one of the composition embodiments described herein, before, simultaneously, or subsequently to the administration of an agent selected from bedaquiline, or a pharmaceutically acceptable salt of derivative thereof, cefoxitine, amikacin, clarithromycin, pyrazinamide, rifampin, moxifloxacin, levofloxacin, and para-amino salicylate, and mixtures thereof. In another embodiment, the agent is bedaquiline or amikacin. In a further embodiment, the agent is bedaquiline.

### Particle Size and Distribution

The therapeutic effect of aerosolized therapies is dependent upon the dose deposited and its distribution. Aerosol particle size is one of the important variables in defining the dose deposited and the distribution of drug aerosol in the lung.

Generally, inhaled aerosol particles are subject to deposition by one of two mechanisms: impaction, which usually predominates for larger aerosol particles, and sedimentation, which is prevalent for smaller aerosol particles. Impaction occurs when the momentum of an inhaled aerosol particle is large enough that the particle does not follow the air stream and encounters a physiological surface. In contrast, sedimentation occurs primarily in the lower lung when very small aerosol particles which have traveled with the inhaled air stream encounter physiological surfaces as a result of gravitational settling.

Pulmonary drug delivery may be accomplished by inhalation of an aerosol through the mouth and throat. Aerosol particles having an aerodynamic diameter of greater than about 5 μm generally do not reach the lung; instead, they tend to impact the back of the throat and are swallowed and possibly orally absorbed. Aerosol particles having diameters of about 3 to about 5 μm are small enough to reach the upper- to mid-pulmonary region (conducting airways), but are too large to reach the alveoli. Smaller aerosol particles, i.e. about 0.5 to about 3 μm, are capable of reaching the alveolar region. Aerosol particles having diameters smaller than about 0.5 μm tend to be exhaled during tidal breathing, but can also be deposited in the alveolar region by a breath hold.

Aerosols used in pulmonary drug delivery are made up of a wide range of aerosol particle sizes, so statistical descriptors are used. Aerosols used in pulmonary drug delivery are typically described by their mass median diameter (MMD), that is, half of the mass is contained in aerosol particles larger than the MMD, and half the mass is contained in aerosol particles smaller than the MMD. For particles with uniform density, the volume median diameter (VMD) can be used interchangeably with the MMD. Determinations of the VMD and MMD are made by laser diffraction. The width of the distribution is described by the geometric standard deviation (GSD). However, the deposition of an aerosol particle in the respiratory tract is more accurately described by the particle's aerodynamic diameter, thus, the mass median aerodynamic diameter is typically used. MMAD determinations are made by inertial impaction or time of flight measurements.

Nonetheless, for the purpose of the description, the aerosol particle size of the aerosol particles will be given as MMAD as determined by measurement at room temperature with a Next Generation Impactor (NGI) in accordance with US Pharmacopeial Convention. In Process Revision <601>Aerosols, Nasal Sprays, Metered-Dose Inhalers, and Dry Powder Inhalers, Pharmacopeial Forum (2003), Volume Number 29, pages 1176-1210 also disclosed in Jolyon Mitchell, Mark Nagel “Particle Size Analysis of Aerosols from Medicinal Inhalers”, KONA Powder and Particle Journal (2004), Volume 22, pages 32-65.

In accordance with the present invention, the particle size of the aerosol is optimized to maximize the deposition of clofazimine at the site of infection and to maximize tolerability. Aerosol particle size may be expressed in terms of the mass median aerodynamic diameter (MMAD). Large particles (e.g., MMAD>5 μm) tend to deposit in the extrathoracic and upper airways because they are too large to navigate bends in the airways.

Intolerability (e.g., cough and bronchospasm) may occur from upper airway deposition of large particles.

Thus, in accordance with a preferred embodiment, the MMAD of the aerosol should be less than about 5 μm, preferably between about 1 and 5 μm, more preferably below 3 μm (<³ μm).

However, a guided breathing maneuver can be used to allow larger particles to pass through the extrathoracic and upper airways and deeper into the lungs than during tidal breathing which will increase the central and lower lung deposition of the aerosol. A guided 10 breathing maneuver may be as slow as 100 ml/min. Thus, when used with a guided breathing maneuver, the preferred MMAD of the aerosol should be less than about 10 μm.

Use in Treatment and/or Prophylaxis

The pharmaceutical compositions and pharmaceutical combinations (aerosols, aerosolized formulations) and systems according to the present invention are intended for the use in the treatment and/or prophylaxis of pulmonary infections caused by mycobacteria or other clofazimine susceptible bacteria, such as Staphylococcus aureus (including methicillin-resistant and vancomycin intermediate-resistant strains), Streptococcus pneumoniae, and Enterococcus spp. The pharmaceutical compositions and pharmaceutical combinations of the present invention may also be used for the treatment and/or prophylaxis of pulmonary fungal infections.

### Dosing of Clofazimine

The daily lung dose (i.e. the dose deposited in the lung) of clofazimine to be administered in accordance with the present invention is about 10-20 mg in the case of M. abscessus infections.

Depending on the dosing frequency, once or twice per day, the daily lung dose will be split accordingly.

In accordance with the present invention, clofazimine is to be administered once or twice daily with a resulting total daily lung dose of about 10 to 20 mg.

It will be obvious to a person skilled in the art that the above amounts relate to clofazimine free base, the dosage amounts for derivatives, and salts will have to be adjusted accordingly based on the MIC of the respective compound and strain.

Accordingly, in a first aspect of the present invention, a pharmaceutical composition is provided, for dry powder inhalation comprising clofazimine, or a pharmaceutically acceptable salt or derivative thereof, of an appropriate particle size, and a physiologically acceptable pharmacologically inert excipient, or a mixture of physiologically acceptable pharmacologically inert excipients of appropriate particle size or sizes.

In a second aspect, a pharmaceutical composition for dry powder inhalation is provided, comprising clofazimine of an appropriate particle size, and a physiologically acceptable pharmacologically inert solid carrier, the solid carrier comprising a physiologically acceptable pharmacologically inert excipient, or a mixture of physiologically acceptable pharmacologically inert excipients of appropriate particle size or sizes.

In a third aspect, a pharmaceutical composition according to the second aspect is provided, wherein the clofazimine is provided in a polymorphic form or forms selected from triclinic form FI, monoclinic form FII and orthorhombic form FIII and mixtures of such forms.

In a fourth aspect of the present invention, a pharmaceutical composition according to the third aspect is provided, wherein the clofazimine is provided substantially in orthorhombic form FIII.

In a fifth aspect, a pharmaceutical composition according to any of the first through fourth aspects is provided, wherein the solid carrier is selected from glucose, arabinose, maltose, saccharose, dextrose and lactose and combinations thereof.

In a sixth aspect, a pharmaceutical composition according to any of the first through fifth aspects, wherein the solid carrier is provided in the form of coarse particles having a mass median diameter of between 50 and 500 μm.

In a seventh aspect, a composition according to any of the first through sixth aspects is provided, wherein the clofazimine is provided in the form of finely divided particles having a mass median aerodynamic diameter of less than 5 μm

In an eighth aspect, a composition according to the seventh aspect is provided, wherein the clofazimine is provided in the form of finely divided particles having a mass median aerodynamic diameter of between 1 μm and 3 μm.

In a ninth aspect, a composition according to any one of the first through fourth aspects is provided, wherein the particles are of homogeneous composition and wherein the particles comprise both clofazimine and the excipient or excipients.

In a tenth aspect, a composition according to the ninth aspect is provided, wherein the particles have a mass median aerodynamic diameter of less than 5 μm.

In an eleventh aspect, a composition according to either of the ninth or tenth aspects is provided, wherein the particles have a mass median aerodynamic diameter of between 1 μm and 3 μm.

In a twelfth aspect, a composition according to any of the first through eleventh aspects is provided, wherein the excipients comprise a phospholipid or a combination of phospholipids.

In a thirteenth aspect, a composition according to any of the first through eleventh aspects is provided, wherein the excipients comprise a salt.

In a fourteenth aspect, a composition according to any one of the first through eleventh aspects is provided, wherein the excipients comprise an amino acid or a combination of amino acids.

In a fifteenth aspect, a composition according to any one of the first through eleventh aspects is provided, wherein the excipients comprise a sugar or combination of sugars.

In a sixteenth aspect, a pharmaceutical combination is provided, comprising a dry powder inhalation device, the dry powder composition according to any one of the first through fifteenth aspects, and a means for introducing the inhalable dry powder composition into the airways of a patient by inhalation.

In a seventeenth aspect, a pharmaceutical combination according to the sixteenth aspect is provided, wherein the dry powder inhalation device is a single dose, or a multi-dose inhaler.

In an eighteenth aspect, a pharmaceutical combination according to the sixteenth aspect is provided, wherein the dry powder inhalation device is pre-metered or device-metered.

In a nineteenth aspect, pharmaceutical composition according to any one of the first through fifteenth aspects is provided, for use in the treatment and/or prophylaxis of pulmonary infections caused by mycobacteria or other gram-positive bacteria.

In a twentieth aspect, a pharmaceutical combination according to any of claims sixteenth through eighteenth aspects is provided, for use in the treatment and/or prophylaxis of pulmonary infections caused by mycobacteria or other gram-positive bacteria.

In a twenty-first aspect, a pharmaceutical composition for use according to the nineteenth aspect is provided, wherein the infection is caused by a species of the genus mycobacterium selected from nontuberculous mycobacteria and Mycobacterium tuberculosis complex, and a combination thereof.

In a twenty-second aspect, a pharmaceutical combination for use according to the twentieth aspect is provided, wherein the infection is caused by a species of the genus mycobacterium selected from nontuberculous mycobacteria and Mycobacterium tuberculosis complex, and a combination thereof.

In a twenty-third aspect, a pharmaceutical composition according to the twenty-first aspect is provided, wherein the nontuberculous mycobacteria is selected from Mycobacterium avium, Mycobacterium intracellulare, Mycobacterium abscessus, and Mycobacterium leprae, and a combination thereof.

In a twenty-fourth aspect, a pharmaceutical combination for use according to the twenty-second aspect is provided, wherein the nontuberculous mycobacteria is selected from Mycobacterium avium, Mycobacterium intracellulare, Mycobacterium abscessus, and Mycobacterium leprae, and a combination thereof.

In a twenty-fifth aspect, a pharmaceutical composition for use according to the twenty-first aspect is provided, wherein the infection is an opportunistic infection, selected from MAC pulmonary disease and nontuberculosis infection, in a patient with cystic fibrosis, chronic obstructive pulmonary disease or acquired immune deficiency syndrome.

In a twenty-sixth aspect, a pharmaceutical combination for use according to the twenty-second aspect is provided, wherein the infection is an opportunistic infection, selected from MAC pulmonary disease and nontuberculosis infection, in a patient with cystic 30 fibrosis, chronic obstructive pulmonary disease or acquired immune deficiency syndrome.

In a twenty-seventh aspect, a pharmaceutical composition according to the twenty-fifth aspect is provided, wherein the infection is an opportunistic nontuberculosis mycobacteria infection in a patient with cystic fibrosis.

In a twenty-eighth aspect, a pharmaceutical combination according to the twenty-sixth aspect is provided, wherein the infection is an opportunistic nontuberculosis mycobacteria infection in a patient with cystic fibrosis.

In a twenty-ninth aspect, a system for use in providing antibiotic activity when treating or providing prophylaxis against a pulmonary infection caused by mycobacteria or other gram-positive bacteria, wherein the system comprises: 1) a dry powder pharmaceutical formulation comprising a) a therapeutically effective dose of clofazimine, b) one or more excipients selected from sugars, amino acids, and phospholipids, and combinations thereof, 2) a container for the formulation selected from a capsule or blister package, and 3) a dry powder inhaler, wherein the clofazimine is present in the form of a dry powder, and wherein the clofazimine containing particles have a mass median diameter of 1 to 5 μm.

In a thirtieth aspect, A pharmaceutical composition according to any of the nineteenth, twenty-first, twenty-third, twenty-fifth, or twenty-seventh aspects is provided, wherein the composition is administered before, simultaneously or subsequently to the administration of an agent selected from bedaquiline or a pharmaceutically acceptable salt or derivative thereof, cefoxitine, amikacin, clarithromycin, pyrazinamide, rifampin, moxifloxacin, levofloxacin, and para-amino salicylate, and mixtures thereof.

In a thirty-first aspect, a pharmaceutical combination according to any of the twentieth, twenty-second, twenty-fourth, twenty-fifth or twenty-eighty aspects is provided, wherein the pharmaceutical combination is used to administer before, simultaneously or subsequently to the administration of an agent selected from bedaquiline or a pharmaceutically acceptable salt or derivative thereof, cefoxitine, amikacin, clarithromycin, pyrazinamide, rifampin, moxifloxacin, levofloxacin, and para-amino salicylate, and mixtures thereof.

In a thirty-second aspect, a pharmaceutical composition according to the thirtieth aspect is provided, wherein the agent is bedaquiline.

In a thirty-third aspect, a pharmaceutical combination according to the thirty-first aspect is provided, wherein the agent is bedaquiline.

In a thirty-fourth aspect, pharmaceutical composition according to the thirtieth aspect is provided, wherein the agent is amikacin.

In a thirty-fifth aspect, a pharmaceutical combination according to the thirtieth aspect is provided, wherein the agent is amikacin.

In a thirtieth-sixth aspect, a method of treatment or prophylaxis of a pulmonary infection caused by mycobacteria or other gram-positive bacteria, in a patient in need thereof, is provided, comprising administering by inhalation a composition according to any one of the first through fifteenth aspects.

In a thirty-seventh aspect, a method of treatment or prophylaxis according to the thirty-sixth aspect is provided, wherein the infection is caused by a species of the genus mycobacterium selected from nontuberculous mycobacteria and Mycobacterium tuberculosis complex, and a combination thereof.

In a thirty-eighth aspect, a method of treatment or prophylaxis according to the thirty-seventh aspect is provided, wherein the nontuberculosis mycobacterium is selected from Mycobacterium avium, Mycobacterium intracellulare, Mycobacterium abscessus, and Mycobacterium leprae, and a combination thereof.

In a thirty-ninth aspect, a method of treatment or prophylaxis according to the thirty-sixth aspect is provided, wherein the infection is an opportunistic infection, selected from MAC pulmonary disease and nontuberculosis infection, in a patient with cystic fibrosis, chronic obstructive pulmonary disease or acquired immune deficiency syndrome.

In a fortieth aspect, a method of treatment or prophylaxis according to the thirty-ninth aspect is provided, wherein the infection is an opportunistic nontuberculosis mycobacteria infection in a patient with cystic fibrosis.

In a forty-first aspect, a method of treatment or prophylaxis of a pulmonary infection caused by mycobacteria or other gram-positive bacteria, in a patient in need thereof, is provided, comprising administering by inhalation a composition according to any one of the first through fifteenth aspects, before, simultaneously, or subsequently to the administration of an agent selected from bedaquiline or a pharmaceutically acceptable salt or derivative thereof, cefoxitine, amikacin, clarithromycin, pyrazinamide, rifampin, moxifloxacin, levofloxacin, and para-amino salicylate, and mixtures thereof.

In a forty-second aspect, a method of treatment or prophylaxis according to the forty-first aspect is provided, wherein the agent is bedaquiline or amikacin.

In a forty-third aspect, a method of treatment or prophylaxis according to the forty-second aspect is provided, wherein the agent is bedaquiline.

### EXAMPLE 1

### Aerolizer DPI

One embodiment of the invention uses the Aerolizer DPI, an inhaler with the drug stored in a capsule. Clofazimine is micronized via jet mill to particles with an MMD of less than 2 μm, then blended with larger lactose particles (MMD greater than 50 μm) to form the formulation. The formulation is approximately 30% clofazimine by weight. Approximately 250 mg of formulation (75 mg clofazimine) is filled into the capsule. Between 13% and 28% of the dose will deposit in the lungs, so this embodiment will deliver between 9.75 mg and 21 mg of clofazimine to the lungs.

We claim:

1. A pharmaceutical composition for dry powder inhalation comprising clofazimine, or a pharmaceutically acceptable salt or derivative thereof, of an appropriate particle size, and a physiologically acceptable pharmacologically inert excipient, or a mixture of physiologically acceptable pharmacologically inert excipients of appropriate particle size or sizes.

2. A pharmaceutical composition for dry powder inhalation comprising clofazimine of an appropriate particle size, and a physiologically acceptable pharmacologically inert solid carrier, the solid carrier comprising a physiologically acceptable pharmacologically inert excipient, or a mixture of physiologically acceptable pharmacologically inert excipients of appropriate particle size or sizes.

3. A pharmaceutical composition according to claim 2 wherein the clofazimine is provided in a polymorphic form or forms selected from triclinic form FI, monoclinic form Fli and orthorhombic form Fill and mixtures of such forms.

4. A pharmaceutical composition according to claim 3 wherein the clofazimine is provided substantially in orthorhombic form Fill.

5. A composition according to claim 2, wherein the solid carrier is selected from glucose, arabinose, maltose, saccharose, dextrose and lactose and combinations thereof.

6. A composition according to claim 2, wherein the solid carrier is provided in the form of coarse particles having a mass median diameter of between 50 and 500 μm.

7. A composition according to claim 1, wherein the clofazimine is provided in the form of finely divided particles having a mass median aerodynamic diameter of less than 5 μm

8. A composition according to claim 7, wherein the clofazimine is provided in the form of finely divided particles having a mass median aerodynamic diameter of between 1 μm and 3 μm.

9. A composition according to claim 1 wherein the particles are of homogeneous composition and wherein the particles comprise both clofazimine and the excipient or excipients.

10-11. (canceled)

12. A composition according to claim 1, wherein the excipients comprise a phospholipid or a combination of phospholipids.

13. A composition according to claim 1, wherein the excipients comprise a salt.

14. A composition according to claim 1, wherein the excipients comprise an amino acid or a combination of amino acids.

15. A composition according to claim 1, wherein the excipients comprise a sugar or combination of sugars.

16. A pharmaceutical combination comprising a dry powder inhalation device, the dry powder composition according to claim 1, and a means for introducing the inhalable dry powder composition into the airways of a patient by inhalation.

17. A pharmaceutical combination according to claim 16, wherein the dry powder inhalation device is a single dose, or a multi-dose inhaler.

18. A pharmaceutical combination according to claim 16, wherein the dry powder inhalation device is pre-metered or device-metered.

19. A pharmaceutical composition according to claim 1, for use in the treatment and/or prophylaxis of pulmonary infections caused by mycobacteria or other gram-positive bacteria.

20-28. (canceled)

29. A system for use in providing antibiotic activity when treating or providing prophylaxis against a pulmonary infection caused by mycobacteria or other gram-positive bacteria, wherein the system comprises:
1) a dry powder pharmaceutical formulation comprising
a) a therapeutically effective dose of clofazimine,
b) one or more excipients selected from sugars, amino acids, and phospholipids, and combinations thereof,
2) a container for the formulation selected from a capsule or blister package, and
3) a dry powder inhaler,
wherein the clofazimine is present in the form of a dry powder, and wherein the clofazimine containing particles have a mass median diameter of 1 to 5 μm.

30. A pharmaceutical composition according to claim 19, wherein the composition is administered before, simultaneously or subsequently to the administration of an agent selected from bedaquiline or a pharmaceutically acceptable salt or derivative thereof, cefoxitine, amikacin, clarithromycin, pyrazinamide, rifampin, moxifloxacin, levofloxacin, and para-amino salicylate, and mixtures thereof.

31. (canceled)

32. A pharmaceutical composition according to claim 30 wherein the agent is bedaquiline.

33. (canceled)

34. A pharmaceutical composition according to claim 30 wherein the agent is amikacin.

35. (canceled)

36. A method of treatment or prophylaxis of a pulmonary infection caused by mycobacteria or other gram-positive bacteria, in a patient in need thereof, comprising administering by inhalation a composition according to claim 1.

37-43. (canceled)
